(19) United States
(12) Patent Application Publication
(10) Pub. No.: US 2021/0183039 A1
(43) Pub. Date: Jun. 17, 2021

(54) SYSTEM AND METHOD OF DEBRIS DETECTION AND INTEGRITY VALIDATION FOR RIGHT-OF-WAY BASED INFRASTRUCTURE

(71) Applicant: LINDSEY FIRESENSE, LLC, Azusa, CA (US)

(72) Inventors: Keith E. Lindsey, La Cañada Flintridge, CA (US); John McCall, Rancho Cucamonga, CA (US); Thomas Lu, Bradbury, CA (US)

(21) Appl. No.: 17/121,722

(22) Filed: Dec. 14, 2020

Related U.S. Application Data
(60) Provisional application No. 62/948,071, filed on Dec. 13, 2019.
(60) Provisional application No. 62/948,078, filed on Dec. 13, 2019.
(60) Provisional application No. 63/067,169, filed on Aug. 18, 2020.

(51) Int. Cl. G06T 7/00; H04N 5/247; H04N 7/18; G06K 9/62; G01N 33/00; G01J 5/10; G01J 5/00; G06N 3/08

(52) U.S. Cl. CPC G06T 7/0002; H04N 5/247; H04N 7/181; G06K 9/6215; G01N 33/0039; G10L 25/51; G01J 5/0018; G06N 3/08; G06T 2207/30184; G06T 2207/20081; G06T 2207/20084; G01J 5/10

(57) ABSTRACT

Systems and methods for debris detection and integrity validation for right-of-way based infrastructures using a neural network are provided. Further, systems and methods for detection of electrical arcs and systems and methods for fire detection using a neural network are provided.

### CROSS-REFERENCE TO RELATED APPLICATIONS

This application claims the benefit of U.S. Provisional Application No. 62/948,071, filed on Dec. 13, 2019, U.S. Provisional Application No. 62/948,078, filed on Dec. 13, 2019, and U.S. Provisional Application No. 63/067,169, filed on Aug. 18, 2020, in the United States Patent and Trademark Office, the entire contents of all of which are incorporated herein by reference.

### BACKGROUND

### 1. Field

Aspects of embodiments of the present invention relate to system and method of debris detection and integrity validation for right-of-way based infrastructure.

### 2. Description of the Related Art

In recent years, the reliability of services provided by right-of-way (ROW) based infrastructure such as power lines, pipelines, railroad lines, and/or the like has become increasingly difficult to maintain as existing infrastructure ages, expands, and is exposed to a variety of environmental conditions. Generally, to restore an existing service, operators, technicians, engineers, and/or the like may diagnose and resolve problems, and perform safety checks.

However, diagnosing and resolving problems, and performing safety checks may be difficult and time-consuming if information regarding the ROW-based infrastructure relies solely on the perspective of on-site workers. Remote inspection techniques, for example through the use of camera equipped drones, are also time-consuming and suffer from ease of comparison to pre-outage conditions. Further, incomplete information based on the perception of the workers may lead to mistakes or errors that may threaten the health and safety of the workers and/or the public while resulting in further delays of service.

The above information disclosed in this Background section is for enhancement of understanding of the background of the present disclosure, and therefore, it may contain information that does not constitute prior art.

### SUMMARY

According to an aspect of one or more embodiments of the present disclosure, systems and methods for debris detection and integrity validation for ROW-based infrastructures are provided.

According to another aspect of one or more embodiments of the present disclosure, an imaging device for capturing “before” and “after” image sets of portions of an object of interest under a variety of conditions is provided.

According to another aspect of one or more embodiments of the present disclosure, systems and methods of reviewing image data sets from one or more imaging devices via a user interface on an electronic device are provided.

According to another aspect of one or more embodiments of the present disclosure, systems and methods for detection of electrical arcs associated with utility electrical equipment are provided.

According to another aspect of one or more embodiments of the present disclosure, systems and methods for fire detection are provided.

According to another aspect of one or more embodiments of the present disclosure, systems and methods for detection of the above-described conditions using a neural network are provided.

### BRIEF DESCRIPTION OF THE DRAWINGS

The above and other features and aspects will become more apparent to those of ordinary skill in the art by describing in further detail some example embodiments of the present invention with reference to the attached drawings, in which:

FIG. 1A is a block diagram of an imaging device according to one or more embodiments of the present disclosure.

FIG. 1B is a block diagram of an electronic communication system including one or more imaging devices according to one or more embodiments of the present disclosure.

FIG. 2A is a perspective view of an imaging device according to one or more embodiments of the present disclosure.

FIG. 2B is a perspective view including blocks indicating components of an imaging device according to one or more embodiments of the present disclosure.

FIG. 3 is a view of a user interface provided to an electronic device available to a user according to one or more embodiments of the present disclosure.

FIG. 4 is a perspective view of a device for detection of electrical arcs according to one or more embodiments of the present disclosure.

FIG. 5 is a perspective view of a device for fire detection according to one or more embodiments of the present disclosure.

FIGS. 6 to 8 are flowcharts illustrating detection methods using a neural network.

### DETAILED DESCRIPTION

Herein, some example embodiments will be described in further detail with reference to the accompanying drawings, in which like reference numbers refer to like elements throughout. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and, thus, descriptions thereof may not be repeated.

In the drawings, relative sizes of elements, layers, and regions may be exaggerated and/or simplified for clarity.

It is to be understood that, although the terms “first,” “second,” “third,” etc., may be used herein to describe various elements, components, regions, layers and/or sections, these elements, components, regions, layers, and/or sections are not limited by these terms. These terms are used to distinguish one element, component, region, layer or section from another element, component, region, layer, or section. Thus, a first element, component, region, layer, or section described below could be termed a second element, component, region, layer, or section, without departing from the spirit and scope of the present disclosure.

It is to be understood that when an element or layer is referred to as being “on,” “connected to,” or “coupled to” another element or layer, it may be directly on, connected to, or coupled to the other element or layer, or one or more intervening elements or layers may be present.

The terminology used herein is for the purpose of describing particular embodiments and is not intended to be limiting of the present disclosure. As used herein, the singular forms “a” and “an” are intended to include the plural forms as well, unless the context clearly indicates otherwise. It is to be further understood that the terms “comprises,” “comprising,” “includes,” and “including,” “has,” “have,” and “having,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which the present disclosure belongs. It is to be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Generally, prior to restarting ROW-based infrastructures that have previously been temporarily removed from service, it may be desirable to perform safety checks and confirm that any problems that may cause or have caused failure of the ROW-based infrastructure have been addressed. However, because ROW-based infrastructure are often very lengthy and meandering in nature, operators, technicians, engineers, and/or the like, may not be aware of the status of the entire ROW-based infrastructure and may not be aware of the previous operational condition of the infrastructure which may be helpful for assessing the current condition of the infrastructure. Time consuming physical or drone-based inspections of the entire ROW infrastructure may be required.

According to one or more embodiments of the present disclosure, an imaging device is provided which captures “before” images and/or video sequences for comparison with “after” images and/or video sequences. Based on the comparison, users such as operators, technicians, engineers, and/or the like may be better able to determine, for example, whether to re-energize an electric power line that has been de-energized. For example, in the case of an electric power line, the users may be able to determine that the power line is both intact (e.g., it has not broken and fallen to the ground) and is not fouled by debris (e.g., tree branches) that would cause an electrical fault upon re-energization.

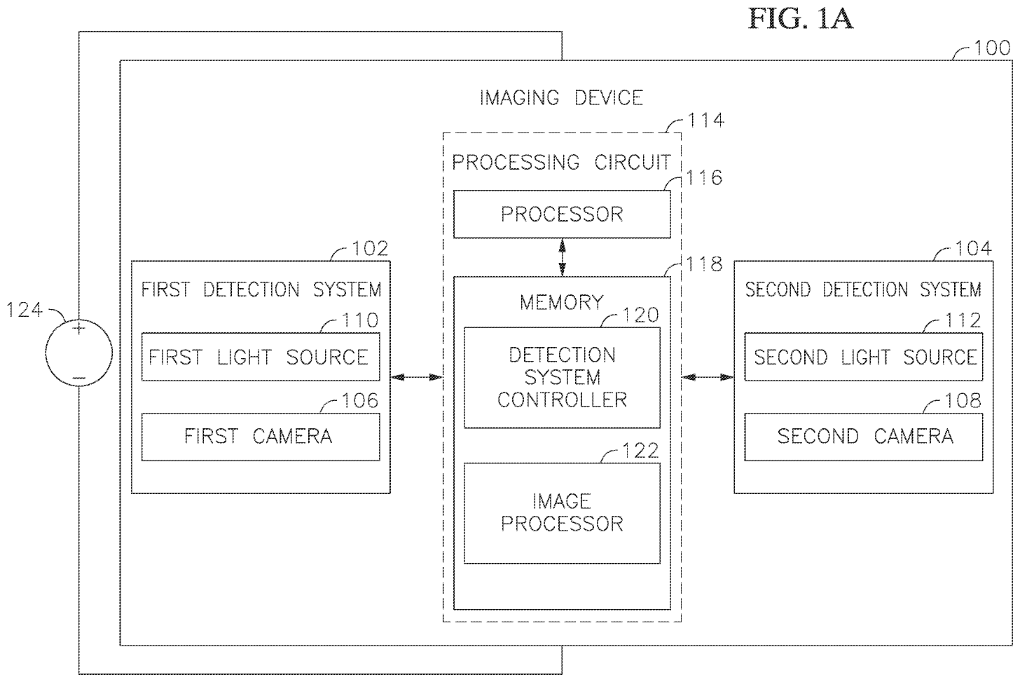

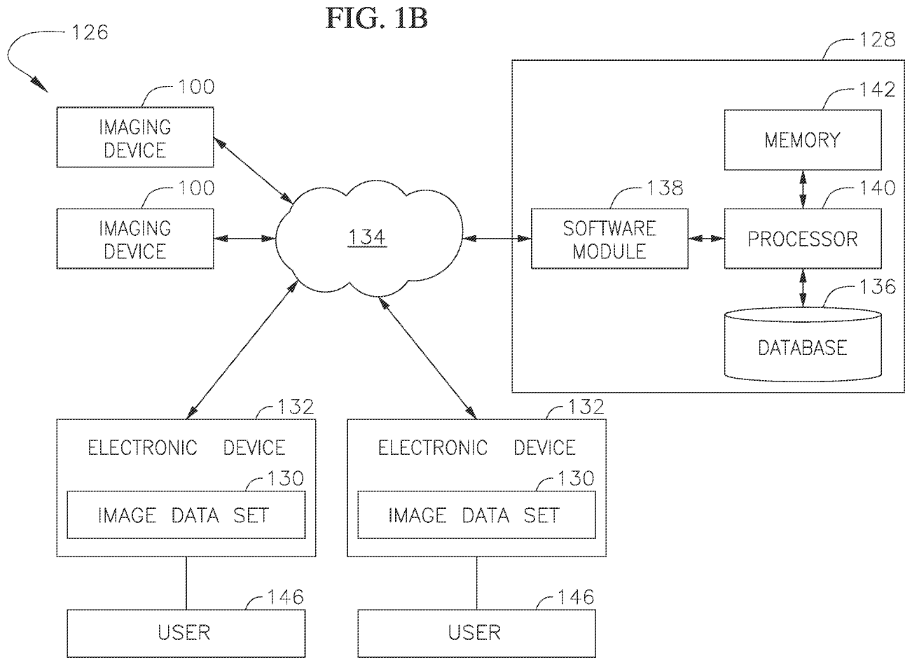

FIG. 1A is a block diagram of an imaging device 100 according to one or more embodiments of the present disclosure.

Referring to FIG. 1A, according to one or more example embodiments, an imaging device 100 includes a first detection system 102 configured to capture images of an environment surrounding the imaging device 100, and a second detection system 104 configured to capture images of an environment surrounding the imaging device 100. As used herein, “images” may refer to images, video sequences, and/or any other suitable format.

Each of the first detection system 102 and the second detection system 104 may be a camera imaging system including one or more cameras 106, 108 coupled to the exterior of or housed with the imaging device 100. The one or more cameras 106, 108 may be configured to capture still and/or video images. The one or more cameras 106 of the first detection system 102 and the one or more cameras 108 of the second detection system 104 may capture overlapping images from the same or different perspectives to create a single, merged image of one or more areas of interest. Third, fourth, or nth detection systems similar to 102 and 104 may be included to match a particular ROW infrastructure.

In one or more embodiments, the one or more areas of interest may include one or more objects of interest such as, for example, portions of a power line and/or components attached to the power line. However, the present disclosure is not limited thereto, and, in other embodiments, areas of interest and associated objects of interest may be areas and objects of other ROW-based infrastructures, such as pipelines, railroad lines, and/or the like.

In one or more embodiments, the first detection system 102 may be facing a first direction, and the second detection system 104 may be facing a second direction opposite to the first direction. Therefore, the first detection system 102 and the second detection system 104 of the imaging device 100 may capture images in, for example, a forward direction and a rearward direction. In this case, the first detection system 102 and the second detection system 104 may capture images of a structure (e.g., a power line, a pipeline, a railroad track, and the like) along a flow direction (e.g., electrical flow, fluid flow, rail transport, and the like). For example the imaging device 100 may be positioned at, on, above, or below a power line such that the first detection system 102 and the second detection system 104 capture images of the power line extending away from opposite ends of the imaging device 100. However, the present disclosure is not limited thereto. For example, in other embodiments, the imaging device 100 may include additional detection systems with one or more cameras set to capture images in any suitable direction desired, such as, for example, a forward direction, a rearward direction, a rightward direction, a leftward direction, a downward direction, an upward direction, and/or the like, such that one or more objects of interest are captured by the imaging device 100 in still and/or video images.

In an embodiment, the first detection system 102 may include a first light source 110 configured to emit light toward a first area of interest (e.g., an area of interest in the first direction) and a first camera 106 configured to detect ambient light (e.g., ambient light including natural light and/or artificial light emitted by, for example, the first light source 110) from the first area of interest. The second detection system 104 may include a second light source 112 configured to emit light toward a second area of interest (e.g., an area in the second direction opposite to the first direction) and a second camera 108 configured to detect ambient light (e.g., ambient light including natural light and/or artificial light emitted by, for example, the second light source 112) from the second area of interest. In one or more embodiments, the first light source 110 and the second light source 112 may be integral with (e.g., housed with) the first camera 106 and the second camera 108, respectively. However, the present disclosure is not limited thereto, and, in other embodiments, the first light source 110 and/or the second light source 112 may be external light sources separate from (e.g., not housed with) the first camera 106 and/or the second camera 108, respectively.

In one or more embodiments, the first light source 110 and the second light source 112 may emit light to facilitate image capture by the first camera 106 and/or the second camera 108, respectively, during low visibility conditions (e.g., nighttime conditions). The first light source 110 and the second light source 112 may emit any suitable wavelength of light for detection by the first camera 106 and the second camera 108, respectively. For example, in one or more embodiments, the first light source 110 and/or the second light source 112 may emit light in the visible wavelength spectrum, and, in other embodiments, the first light source 110 and/or the second light source 112 may emit light in an infrared, ultraviolet, or other non-visible wavelength spectrum. Light in the non-visible wavelength spectrum may be more conducive for detection by the first camera 106 and/or the second camera 108 under certain lighting conditions (e.g., nighttime), physical conditions, weather, and/or expected debris type (e.g., the type of debris that may undesirably affect the integrity of or interfere with operation of the one or more objects of interest).

Although the first light source 110 and the second light source 112 are described with reference to FIG. 1, in one or more embodiments, the first light source 110 and/or the second light source 112 may be omitted. For example, the first light source 110 and/or the second light source 112 may not be included to save power, cost, or to provide a smaller form factor.

In one or more embodiments, the imaging device 100 includes a processing circuit 114 in communication with the first detection system 102 and the second detection system 104. The processing circuit 114 may control the first detection system 102 and the second detection system 104, and may manage storage of video sequences and/or images captured by the first detection system 102 and the second detection system 104.

In one or more embodiments, the processing circuit 114 of the storage device includes a processor 116 and memory 118. The processor 116 may be implemented as a general purpose processor, an application specific integrated circuit (ASIC), one or more field programmable gate arrays (FPGAs), a group of processing components, or any other suitable electronic processing components. The memory 118 (e.g., memory, memory unit, storage device, and/or the like) may include one or more devices (e.g., RAM, ROM, Flash memory, hard disk storage, and/or the like) for storing data and/or computer code for completing or facilitating the various processes described in the present application. The memory 118 may be or include volatile memory or non-volatile memory. The memory 118 may include database components, object code components, script components, or any other type of information structure for supporting the various activities and information structures described in the present application. According to one or more embodiments, the memory 118 may be communicably connected to the processor 116 via the processing circuit 114, and includes computer code for executing (e.g., by the processing circuit 114 and/or the processor 116) one or more processes described herein.

As shown in FIG. 1A, in one or more embodiments, the processing circuit 114 may be implemented within the imaging device 100 as an internal processing circuit of the imaging device 100. However, the present disclosure is not limited thereto. For example, (as indicated by the dotted rectangular block shown in FIG. 1A), the processing circuit 114 or one or more components thereof (e.g., components executing instructions in memory to perform the methods described in the present disclosure) may be distributed across multiple servers or computers that may exist in distributed locations.

In one or more embodiments, the processing circuit 114 may execute instructions in memory 118 to function as a detection system controller 120 and/or an image processor 122. The detection system controller 120 may activate and deactivate the first detection system 102 and/or the second detection system 104 based on set (e.g., predetermined) logic and/or user input via an external signal. The image processor 122 may prepare the images provided by the first detection system 102 and the second detection system 104 for storage and upload to one or more electronic devices 132 (see FIG. 1B) such as, for example, a personal computer, a server, and/or the like.

In one or more embodiments, the detection system controller 120 may be set to activate the one or more cameras of the first detection system 102 and/or the one or more cameras of the second detection system 104 at set times throughout the day to capture images of the first area of interest and/or the second area of interest. The set times throughout the day may be based on the appearance of an object of interest (e.g., a portion of a power line) in the first area of interest and/or the second area of interest under a variety of ambient lighting conditions (e.g., ambient light conditions including natural lighting and/or artificial lighting from a light source).

The images capturing the one or more objects of interest in a desired configuration (e.g., a configuration including an arrangement of the one or more objects of interest operating as desired) may be designated by the image processor 122 as “before” images when storing the storage images in memory 118. For example, images of an operational power line (e.g., an energized power line) may be captured by the imaging device 100 to be used as “before” images. The image processor 122 may store the “before” images with an actual time period and a representative time period. The representative time period may be greater than the actual time period and range from minutes to days depending on the attributes of the object of interest (e.g., the portion of a power line) and the conditions that the object of interest may be subject to, such as lighting conditions (e.g., nighttime), physical conditions, weather, and/or expected debris type (e.g., the type of debris that may affect the integrity of or interfere with operation of the one or more objects of interest).

In one or more embodiments, the detection system controller 120 may deactivate (or turn off) the one or more cameras of the first detection system 102 and the one or more cameras of the second detection system 104 in response to set (e.g., predetermined logic) and/or user input via external signals to avoid capturing “before” images including debris, undesirable conditions, and the like. For example, the one or more cameras of the first detection system 102 and the one or more cameras of the second detection system 104 may be turned off by any suitable mechanism including a communication signal sent to the imaging device 100, a signal from an integral or separate power line current sensor to indicate the line is de-energized, a signal from an integral or separate weather sensor (e.g., a wind speed sensor) that may indicate stormy conditions exist where windborne debris may be present, and/or remote removal of power to the imaging device 100 (e.g., the one or more cameras of the imaging device 100). However, the present disclosure is not limited thereto.

For example, in one or more embodiments, the detection system may not disable the one or more cameras of the first detection system 102 and the one or more cameras of the second detection system 104 in response to adverse conditions (e.g., stormy conditions and the like). In this case, any of the captured images by either detection system may be transmitted to a user for troubleshooting purposes.

If the one or more cameras are deactivated, the detection system controller 120 may activate (or turn on) the one or more cameras of the first detection system 102 and the one or more cameras of the second detection system 104 prior to operating the ROW-based infrastructure. For example, after a power line is de-energized and before a utility re-energizes the power line, the detection system controller 120 may activate the one or more cameras of the first detection system 102 and the one or more cameras of the second detection system 104 to capture new images. The image processor 122 may designate the new images as “after” images when storing the new images in memory 118. In one or more embodiments, the “after” designation may be applied by the image processor 122 in response to user input or being powered on.

In one or more embodiments, the image processor 122 may associate the “before” images with corresponding “after” images based on the actual time period or the representative time period of the “before” images. In other words, the “after” images may be associated with “before” images captured at a similar time of day and/or under similar conditions. The image processor 122 may transmit “before” images with the associated “after” images to a user (e.g., an operator) or a server for later retrieval and longer term storage as described in further detail with reference to FIG. 1B. Accordingly, the user (e.g., the operator) may compare the “before” and “after” images to determine if the comparison indicates a sufficient difference in appearance that would suggest that the integrity of one or more objects of interest has been violated. For example, the integrity of a power line may be violated when, for example, a conductor is broken or fouling debris may be present (e.g., tree branches lying across one or more conductors of the power line).

Although the image processor 122 of the imaging device 100 is described as associating the “before” and “after” images, the present disclosure is not limited thereto. For example, the association may be done manually by a user based on time, date, location data, and the like, or may be performed by the server and/or one or more electronic devices 132 receiving the “before” and “after” images from the imaging device 100.

In one or more embodiments, the imaging device 100 and components thereof may be supplied with power from any suitable power source 124. For example, an external alternating current (AC) or direct current (DC) power source, solar panels, a magnetic field harvesting power supply, and/or the like, and may contain a battery or other source such as a fuel cell to ensure operation for a period of time in the event the power source 124 ceases to function. For example, the battery may provide power at night in conjunction with a solar panel-based power source 124.

FIG. 1B is a block diagram of an electronic communication system 126 including one or more imaging devices 100 according to one or more embodiments of the present disclosure.

Referring to FIG. 1B, the one or more imaging devices 100 may be part of an electronic communication system 126 for processing, communicating, and/or reviewing (e.g., annotating) an image data set 130 including images from the one or more imaging devices 100 according to one or more embodiments of the present disclosure. In an embodiment, the electronic communication system 126 may include a server 128, one or more electronic devices 132 operated by one or more corresponding users 146, and one or more imaging devices 100.

The one or more users 146 may be, for example, operators, technicians, engineers, and/or the like. The one or more users 146 may operate the one or more electronic devices 132 to view images from the one or more imaging devices 100. Depending on the privileges of the one or more users 146, the users 146 may annotate the image data set 130 including images from the one or more imaging devices 100. For example, the one or more users 146 may provide custom notes associated with any of the images, an indication of whether any of the images has been reviewed, and/or an indication of whether any of the images indicates conditions in which an in-person or other suitable inspection (field check) is desired or required to validate whether the ROW infrastructure location requires repair, replacement, restoration, clearing, etc., as annotated by a user 146. Although two electronic devices 132, two imaging devices 100, and one server 128 are shown in FIG. 1B, the present disclosure is not limited thereto. For example, any suitable number of electronic devices 132, imaging devices 100, and/or servers 128 may be communicably connected with each other via the electronic communication system 126.

In one or more embodiments, the server 128 may be connected to (i.e. in electronic communication with) the one or more electronic devices 132 and the one or more imaging devices 100 over a data network 134, such as, for example, a local area network or a wide area network (e.g., a public Internet). The server 128 may include a software module 138 for coordinating electronic communications between the users 146, one or more imaging devices 100, and a database 136 of the server to provide the functions described throughout the application.

In one or more embodiments, the server 128 may include a mass storage device or database 136, such as, for example, a disk drive, drive array, flash memory, magnetic tape, or other suitable mass storage device for storing information used by the server 128. For example, the database 136 may store images, attributes of the images including location data, time, date, designation (e.g., “before,” “after,” or no designation), annotations, and the like. The database 136 may also store imaging device settings, such as camera settings and/or an identification or group associated with one or more imaging devices 100, and the like. The database 136 may also store data associated with any of the image or device attributes, but collected from other sources. For example, the database 136 may store wind speed, wind direction, or other weather data associated with the location of a imaging device 100 as collected from other sensors or third party services at the time an image was captured. Although the database 136 is included in the server 128 as illustrated in FIG. 1B, the present disclosure is not limited thereto. For example, the server 128 may be connected to an external database that is not a part of the server 128, in which case, the database 136 may be used in addition to the external database or may be omitted entirely.

The server 128 may include a processor 140 which executes program instructions from memory 142 to perform the functions of the software module 138. The processor 140 may be implemented as a general purpose processor 140, an application specific integrated circuit (ASIC), one or more field programmable gate arrays (FPGAs), a group of processing components, or other suitable electronic processing components. The memory 142 (e.g., memory, memory unit, storage device, and/or the like) may include one or more devices (e.g., RAM, ROM, Flash memory, hard disk storage, and/or the like) for storing data and/or computer code for completing or facilitating the various processes described for the software module 138. The memory 142 may be or include volatile memory or non-volatile memory. The memory 142 may include database components, object code components, script components, or any other type of information structure for supporting the various activities and information structures described for the software module 138. According to one or more embodiments, the memory 142 may be communicably connected to the processor 140 via the server 128, and may include computer code for executing one or more processes described for the software module 138.

In one or more embodiments, the one or more electronic devices 132 and the one or more imaging devices 100 may be connected to the electronic communication system 126 via a telephone connection, satellite connection, cable connection, radio frequency communication, mesh network, or any other suitable wired or wireless data communication mechanism. In one or more embodiments, the electronic devices 132 may take the form of, for example, a personal computer (PC), hand-held personal computer (HPC), personal digital assistant (PDA), tablet or touch screen computer system, telephone, cellular telephone, smartphone, or any other suitable electronic device.

In one or more embodiments, the image data set 130 may be transmitted to the one or more electronic devices 132 and/or the server 128 upon receipt, by one or more imaging devices 100, of the command or trigger to stop capturing or designating “before” images of the image data set 130. By preemptively transmitting a portion of the image data set 130 (e.g., the “before” images), an image data set 130 including the “before” and “after” images may be more quickly available for review by a user 146 because the one or more imaging devices 100 may only need to transmit the “after” images in response to capturing the “after” images. Accordingly, the one or more imaging devices 100 may transmit the “before” and “after” images of the image data set 130 separately. However, the present disclosure is not limited thereto, and, in other embodiments, the “before” images of the image data set 130 may be sent concurrently with the command or trigger to send “after” images of the image data set 130.

In one or more embodiments, one or more imaging devices 100 may be grouped together as desired. For example, one or more imaging devices 100 viewing or installed on the same power line may be part of a group. The detection system controller 120 of each of the one or more imaging devices 100 of the group may receive a stop command or be triggered to stop capturing or designating “before” and/or “after” images. Upon receipt of the stop command sent to the group or trigger applied to the group, an image data set 130 from each of the one or more imaging devices 100 in the group may be transmitted to the one or more electronic devices 132 and/or server 128. By stopping one group at a time, the user 146 may review the image data sets 130 of one group at a time instead of waiting to receive and review image data sets 130 associated with imaging devices 100 of multiple groups. In other words, by grouping one or more imaging devices 100 according to a set scheme (e.g., by power line), the review process may be sped up because the user 146 may review, for example, one power line at a time instead of waiting for data from imaging devices of multiple groups corresponding to multiple power lines at once.

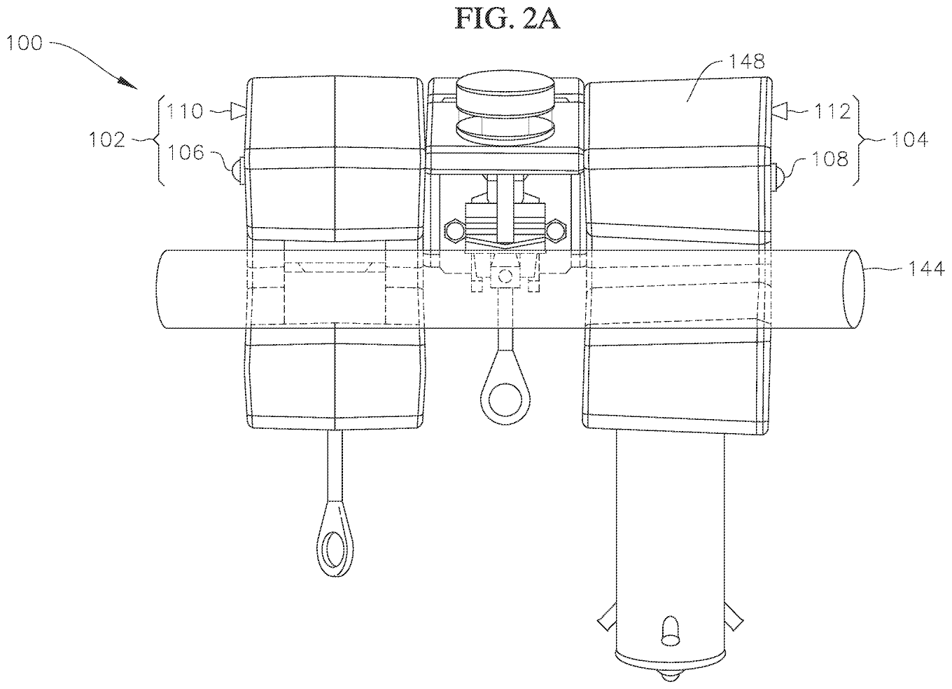

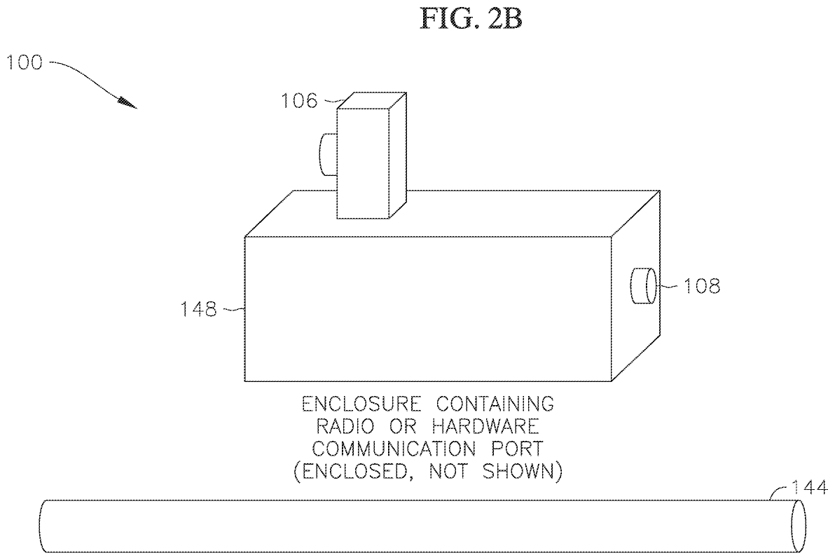

FIG. 2A is a perspective view of an imaging device 100 according to one or more embodiments of the present disclosure.

Referring to FIG. 2A, an imaging device 100 according to one or more embodiments of the present disclosure may include a first detection system 102 and a second detection system 104. The first detection system 102 may include a first camera 106 and a first light source 110, and the second detection system 104 may include a second camera 108 and a second light source 112. In an embodiment, the first camera 106, the first light source 110, the second camera 108, and the second light source 112 may be integral with (e.g., housed with) each other.

As shown in FIG. 2A, in one or more embodiments, the imaging device 100 may include a housing 148 which is mountable on (e.g., directly mountable on) a conductor 144, or power line, such that the first camera 106 and the second camera 108 capture images of the conductor 144 at opposite sides of the imaging device 100. As such, the imaging device 100 may capture “before” and “after” images of the conductor 144. The “before” and “after” images may be transmitted to the electronic device 132 and/or the server 128 through the data network 134 for review and storage, respectively. In one or more embodiments, the housing 148 of the imaging device 100 may accommodate radio or hardware communication circuitry, an integral or external magnetic field harvesting power supply, a solar panel power supply, and/or a battery.

FIG. 2B is a perspective view including blocks indicating components of an imaging device 100 according to one or more embodiments of the present disclosure.

Referring to FIG. 2B, an imaging device 100 according to one or more embodiments of the present disclosure may include a first detection system including a first camera 106 and a second detection system including a second camera 108. The first camera 106 may not be integral with (e.g., may not share a housing with) other components of the imaging device 100. For example, the first camera 106 may be mounted on a surface of a housing 148 enclosing radio or hardware communication circuitry, a solar panel power supply, and/or a battery. In one or more embodiments, the second camera 108 may be integral with (e.g., may share a housing with) the imaging device 100. However, the present disclosure is not limited thereto, and any cameras and/or light sources may be integral with (e.g., housed with) or separate from (e.g., spaced apart from or mounted on a surface of) other components of the imaging device 100.

In one or more embodiments, the first camera 106 and the second camera 108 may be oriented such that the first camera 106 and the second camera 108 capture images of the conductor 144 from opposite sides of the imaging device 100, or at fixed angles with respect to each other, or installed on a locally or remotely adjustable mounting, to better capture images of the conductor 144 at a location (e.g., a location where a power line makes a change in angle to follow its easement). As such, the imaging device 100 may capture “before” and “after” images including portions of the conductor 144. The “before” and “after” images may be transmitted to an electronic device and/or a server for review and storage, respectively.

Although a conductor 144 of a power line is captured by the imaging device 100 in FIGS. 2A and 2B, the present disclosure is not limited thereto. For example, in other embodiments, the imaging device 100 may be used with other ROW-based infrastructures, such as pipelines, railroad lines, and/or the like in a similar manner.

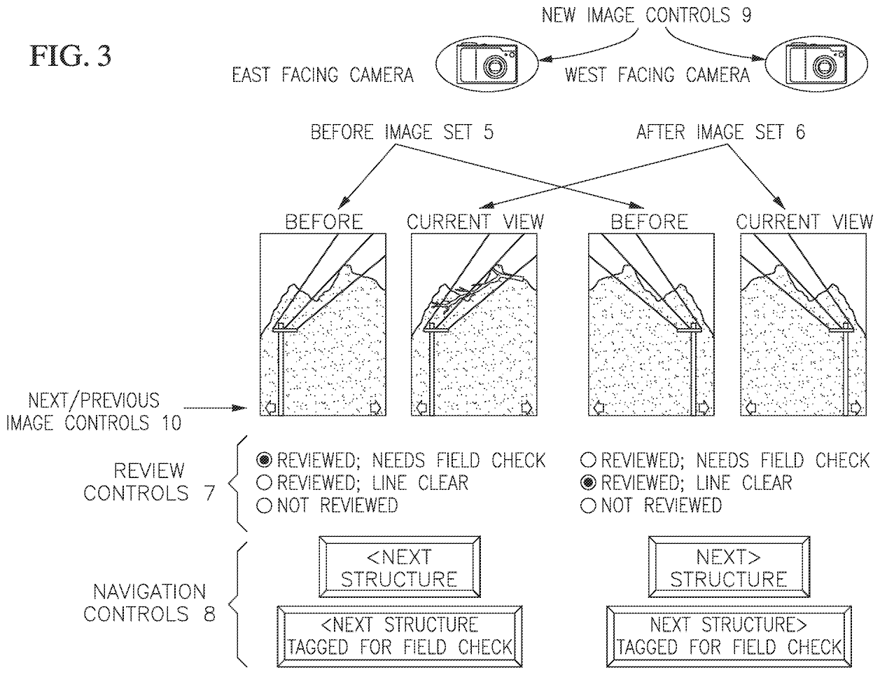

FIG. 3 is a view of a user interface provided to an electronic device 132 available to a user according to one or more embodiments of the present disclosure.

In one or more embodiments, a user 146 may manually view image data sets 130 (see, e.g., FIG. 1B) including images from one or more imaging devices 100 via a user interface. Each image data set 130 may include a “before” image set and an “after” image set based on the designation of “before” or “after” set by the imaging device 100 (e.g., the image processor) capturing the images stored in the image data set. In one or more embodiments, the user interface may be a computer- or internet-based user interface that simplifies the visual comparison of the “before” and “after” image sets.

As shown in FIG. 3, a “before” image set 5 and an “after” image set 6 may be viewed side-by-side for ease of comparison. Controls 10 may allow the user 146 to view images taken previously or later in time from the currently viewed “before” image set 5 and the “after” image set 6. In one or more embodiments, controls 9 may allow the user 146 to capture and transmit new images from the imaging device 100 to be displayed as new “after” images adjacent to the currently viewed “before” image set 5 as desired. In other words, the user 146 may manually operate the first detection system 102 and/or the second detection remotely to capture and transmit new images (e.g., “after” images).

In one or more embodiments, a set of review controls 7 may allow the user 146 to indicate the results of the review (e.g., “reviewed; needs field check,” “reviewed; line clear,” or “not reviewed,” as shown in FIG. 3). In one or more embodiments, navigation controls 8 may allow the user 146 to easily move to other image data sets 130 from another imaging device 100 installed on the next location of the power line (e.g., the same or a different conductor), and/or to the next device 100, which has already been tagged as “needs field check,” and/or a different power line as desired.

Accordingly, as disclosed herein, one or more embodiments of the present disclosure provide an imaging device 100 which captures “before” images for comparison with “after” images. Based on the comparison, users 146, such as operators, technicians, engineers, and/or the like, may be better able to determine, for example, whether to re-energize a power line that has been de-energized.

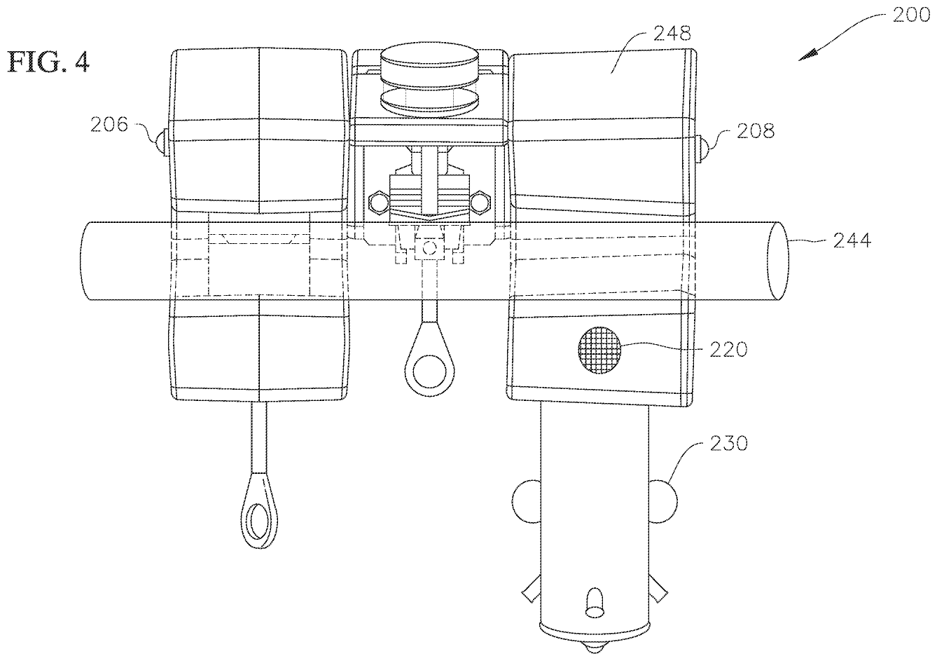

FIG. 4 is a perspective view of a device 200 for detection of electrical arcs according to one or more embodiments of the present disclosure.

Wildfires may be caused by electrical arcs associated with utility electrical equipment. This is often the result of wind-related conductor movement whereby conductors either come in contact with each other, or the movement reduces the electrical clearance between them, or the presence of an animal which reduces the electrical clearance, or between a conductor and its metallic support structure whereby an electrical arc jumps between the conductors or the conductor and the structure, or by an electrical equipment failure. The resulting arc can be blown by the wind and come in contact with a flammable material (e.g., brush, trees, grass, etc.) thereby starting a wildfire. Detection of external environmental phenomena associated with electrical arcs can be used to alert electric utility or fire-fighting personnel of a possible fire. Such detection can also be used to place other wildfire detection sensing equipment into higher alert states (e.g., more frequent sensing cycles or lowered sensing thresholds).

In an embodiment, the device 200 for detection of electrical arcs may include a combination of one or more cameras 206, 208, an RF detector included at a housing 248, one or more microphones 230, and an ozone detector 220. The device 200 may be mounted on a utility power line 244, or installed on a stand-alone structure or support. The various sensor outputs are configured to continuously monitor for the optical signatures associated with electrical arc flashes, the slow front RF waves associated with power frequency arcs, the audio signatures associated with the crackle and buzzing associated with arcs, and an increase in the level of detected ozone, a byproduct of arcs. In an embodiment, the one or more cameras 206, 208, the RF detector, the one or more microphones 230, and the ozone detector 220 may be integral with (e.g., housed with) each other.

In an embodiment, algorithms in an onboard microprocessor provide processing for the suitable arc-related interpretation of each sensor output. Detection of two or more arc-related phenomena will result in the declaration of a possible arc event. This declaration may result in the device 200 to communicate the condition to personnel or entities interested in this condition, including, but not limited to, electric utility and wildfire command center personnel or systems. The declaration may also cause other systems in the device 200 to change an operating state. For example, one or more of the cameras 206, 208 may be triggered to capture images or video and store or transmit the same to interested personnel or systems. Also, in an embodiment, the device 200 may include heat detectors which may be set to poll at a higher frequency in order to detect heat from a fire.

As shown in FIG. 4, in one or more embodiments, the device 200 for detection of electrical arcs may include the housing 248 which is mountable on (e.g., directly mountable on) a conductor 244, or power line. The output from the one or more cameras and sensors may be transmitted to an electronic device and/or a server through a data network for review and storage, respectively. In one or more embodiments, the housing 248 of the device 200 for detection of electrical arcs may accommodate radio or hardware communication circuitry, an integral or external magnetic field harvesting power supply, a solar panel power supply, and/or a battery.

In one or more embodiments, the device 200 for detection of electrical arcs may include a processing circuit that is the same or similar to the processing circuit 114 described above with respect to the imaging device 100. Further, in one or more embodiments, one or more of the device 200 for detection of electrical arcs may be part of an electronic communication system that is the same or similar to the electronic communication system 126 described above with respect to the imaging device 100. Therefore, further description of the processing circuit and the electronic communication system associated with the device 200 for detection of electrical arcs will not be provided.

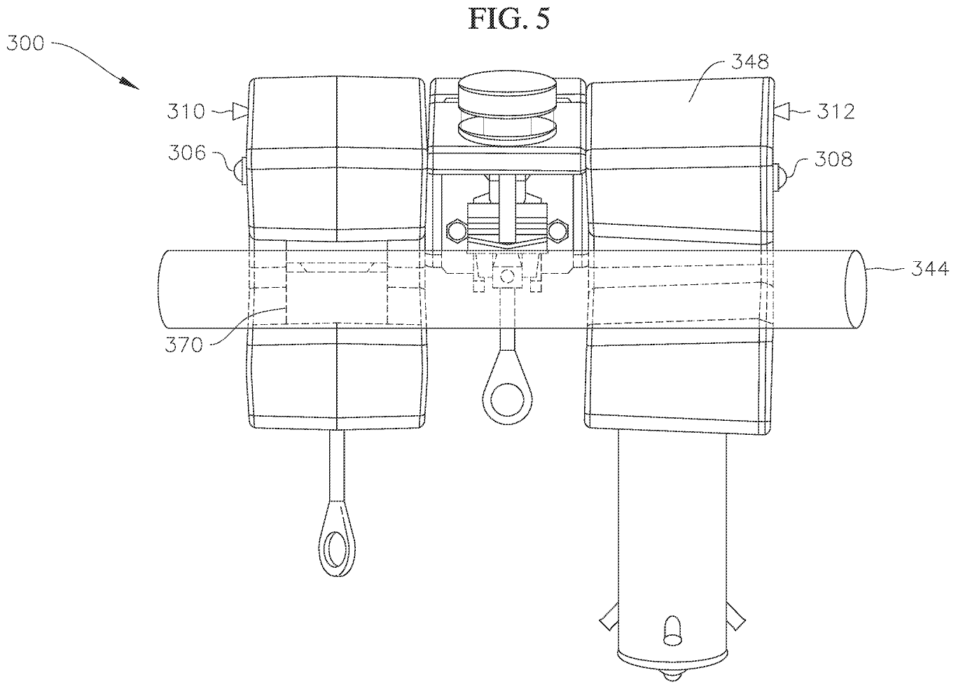

FIG. 5 is a perspective view of a device 300 for fire detection according to one or more embodiments of the present disclosure.

The device 300 for fire detection may be similar to the device 200 for detection of electrical arcs and may include similar components. In an embodiment, the device 300 for fire detection may include one or more cameras 306, 308, one or more infrared (IR) sensors 310, 312, and an external magnetic field harvesting power supply 370, such as to obtain power from a conductor 344, or power line, on which the device 300 for fire detection is mounted. In an example embodiment, the IR sensors may be of a 32×32 array type, and the cameras may be of an 8-megapixel type, but embodiments of the present invention are not limited thereto. In an embodiment, the device 300 for fire detection may also include one or more thermal sensors (e.g., thermopiles). In an embodiment, the one or more cameras, sensor, and other components may be integral with (e.g., housed with) each other.

As shown in FIG. 5, in one or more embodiments, the device 300 for fire detection may include a housing 348 which is mountable on (e.g., directly mountable on) a conductor 344, or power line. The outputs from the one or more cameras, one or more IR sensors, and other sensors may be transmitted to an electronic device and/or a server through a data network for review and storage, respectively. In one or more embodiments, the housing 348 of the device 300 for fire detection may accommodate radio or hardware communication circuitry, an integral or external magnetic field harvesting power supply, a solar panel power supply, and/or a battery.

In one or more embodiments, the device 300 for fire detection may include a processing circuit that is the same or similar to the processing circuit 114 described above with respect to the imaging device 100. In one embodiment, the device 300 for fire detection may include a first microprocessor to receive and process data from the one or more cameras, and a second microprocessor to receive and process data from the one or more IR sensors. Further, in an embodiment, the first microprocessor may obtain and process data from the thermal sensors and may require a lower amount of power than the second microprocessor. In an embodiment, the first microprocessor may be powered by the battery, such as at night. In an embodiment, the second microprocessor may be turned on so as to take and process images when a certain condition is detected by the first microprocessor. Further, in one or more embodiments, one or more of the device 300 for fire detection may be part of an electronic communication system that is the same or similar to the electronic communication system 126 described above with respect to the imaging device 100. Therefore, further description of the processing circuit and the electronic communication system associated with the device 300 for fire detection will not be provided.

Further, while the imaging device 100, the device 200 for detection of electrical arcs, and the device 300 for fire detection have been shown and described separately, in one or more embodiments, one or more of the cameras, sensors, and/or other components of the various embodiments may be combined in a same device.

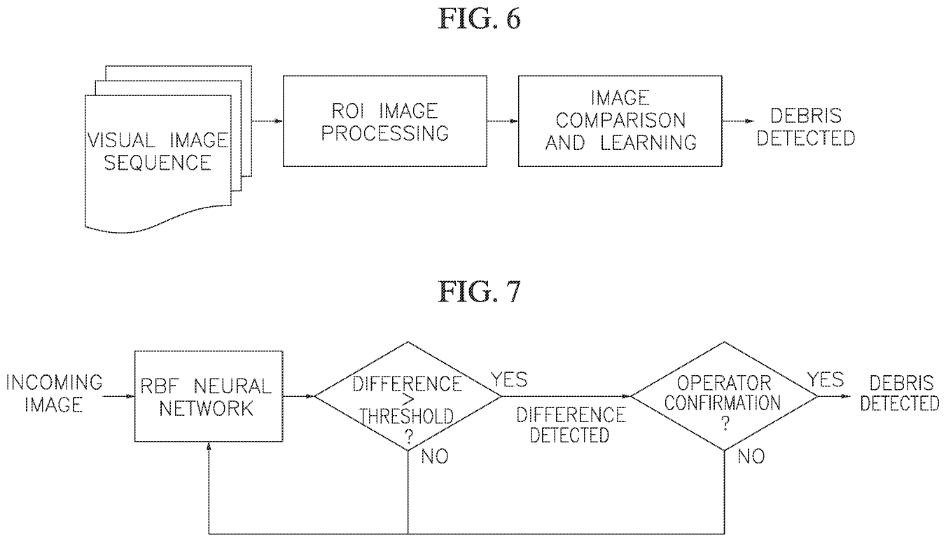

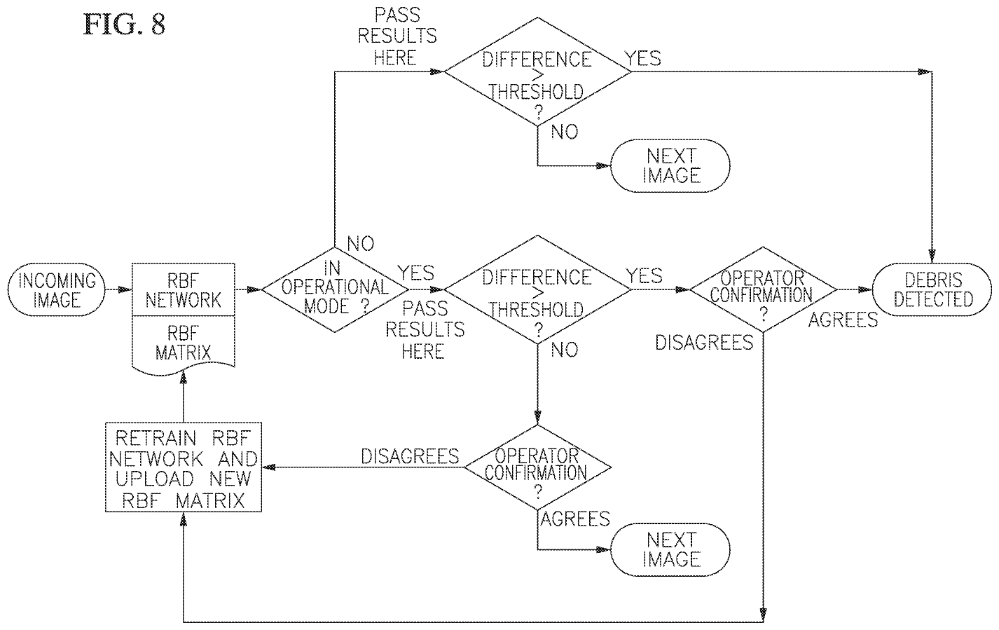

FIGS. 6 to 8 are flowcharts illustrating detection methods using a neural network. According to one or more embodiments, the methods described with respect to FIGS. 6 to 8 may be performed in connection with any of the imaging device 100, the device 200 for detection of electrical arcs, and the device 300 for fire detection described above.

In one or more embodiments, region of interest (ROI) image processing is performed with respect to a visual image sequence. In an embodiment, image pre-processing to clean up incoming images from the one or more cameras may be performed. For example, areas of images may be narrowed to the region of interest defined by a user.

Further, imaging comparison and learning is performed. An incoming image is compared to a reference image in a library of the system. If a difference between the incoming image and the reference image is greater than a threshold value, a condition (e.g., debris is on a power line) is detected. If the difference is less than a threshold value, then the system learns the change and adapts the change into the library. In an embodiment, an image comparison and learning system may be a Radial Bases Function (RBF) neural network, but the present invention is not limited thereto and, in other embodiments, another suitable neural network may be used. The neural network may automatically learn to categorize the incoming image into a most similar category. Further, the neural network compares the incoming image with its neural branches and determines if the new images belongs to an existing branch, or if it is a different image. In an operational mode, the neural network gives a warning that the new image difference may indicate a certain condition (e.g., debris, such as a tree branch, on a power line). In a learning mode of the neural network, if an operator determines that a new image is not indicative of a certain condition (e.g., debris on a power line), then the neural network learns the new image difference into its neural branches.

Further, in one or more embodiments, the neural network may be trained by providing a series of computerized simulations, such as images of debris on a power line. Similarly, in a device for fire detection, images of synthetic fires may be generated and provided to the neural network in the training and building of the library. In one or more embodiments, the neural network looks for changes, rather than looking for any particular signal, and learns on its own to build intelligence. For example, the neural network may learn patterns, and may unlearn, such as when a human operator informs the neural network that a certain condition (e.g., debris on a power line) exists. For example, a number of images (e.g., several hundred images) of different size, location, intensity, etc. may be provided to train the neural network.

In an application for fire detection, a number of background images may be collected, such as background images, day/night images, images from different seasons to be added to the library. Similarly, in the training of the neural network, a number of synthetic images representing different conditions may be input to the library, so as to represent a particular condition of interest, such as debris on a power line or a fire.

In an embodiment, recognition of a certain condition (e.g., debris on a power line, an arc, or a fire) is performed at the device, or, in another embodiment, in the cloud. In an embodiment, the recognition is performed at the device, though the training of the device may be performed from a server at another location due to memory requirements, although it is possible that the training may also be performed at the device, depending on the CPU processing capabilities on the device. In an embodiment, recognition of a certain condition may be performed quickly at the device itself, as compared to a case in which data is sent to the cloud or a remote location for comparison and/or recognition of a condition, particularly when many device are sending data concurrently.

In one or more embodiments, two or more neural networks may be provided in a device, such as a fire detection device. For example, in a fire detection device, one neural network may be trained with respect to thermal data, and another neural network may be trained with respect to optical data. In an embodiment, images collected from multiple devices may be used in training, for example, in creating or updating a matrix to be downloaded to one or more devices. In another embodiment, images collected from a same device over a period of time may be used in training the device.

In one or more embodiments, training of the neural network may be performed as described in SPIE Pattern Recognition and Tracking Conference 10995-18, April 2019, “Optimized training of deep neural network for image analysis using synthetic targets and augmented reality” by Thomas Lu et al. and/or SPIE Defense+Security, Pattern Recognition & Tracking XXIX, Vol. 10649, No. 35, Orlando, Fla., 2018, “Augmented reality data generation for training deep learning neural network” by Keven Payumo et al., the entire contents of both of which are incorporated herein by reference.

Although some example embodiments have been described herein, those skilled in the art will readily appreciate that various modifications are possible in the example embodiments without departing from the spirit and scope of the present disclosure. It is to be understood that descriptions of features or aspects within each embodiment should typically be considered as available for other similar features or aspects in other embodiments, unless otherwise described. Therefore, it is to be understood that the foregoing is illustrative of various example embodiments and is not to be construed as limited to the specific example embodiments disclosed herein, and that various modifications to the disclosed example embodiments, as well as other example embodiments, are intended to be included within the spirit and scope of the present disclosure as set forth in the appended claims, and their equivalents.

What is claimed is:

1. An imaging device comprising:
a first camera configured to capture images of a first portion of an object of interest, the first camera being directed toward a first direction; and
a processing circuit configured to:
receive data from the first camera;
using a neural network, perform a comparison of a first image captured by the first camera at a first time with a first reference image stored in a library of the processing circuit; and
transmit information of the comparison to a user.

2. The imaging device of claim 1, further comprising a second camera configured to capture images of a second portion of the object of interest, the second camera being directed toward a second direction,
wherein the processing circuit is further configured to:
perform a comparison of a second image captured by the second camera at a first time with a second reference image stored in the library of the processing circuit; and
transmit information of the comparison of the second image to a user.

3. The imaging device of claim 1, wherein the processing circuit is further configured to:
perform a comparison of a second image captured by the first camera at a second time with the first reference image stored in the library of the processing circuit; and
transmit information of the comparison of the second image to a user.

4. The imaging device of claim 1, wherein the object of interest is a power line.

5. The imaging device of claim 4, further comprising a magnetic field harvesting power supply configured to obtain power from the power line to power the imaging device.

6. The device of claim 5, further comprising a battery that is chargeable by the power obtained by the magnetic field harvesting power supply.

7. The imaging device of claim 1, wherein the second direction is opposite the first direction.

8. The imaging device of claim 1, further comprising at least one of an RF detector or a microphone, wherein the processing circuit is further configured to receive data from the at least one of the RF detector or the microphone.

9. The imaging device of claim 1, further comprising an ozone detector, wherein the processing circuit is further configured to receive data from the ozone detector.

10. The imaging device of claim 1, further comprising at least one of an infrared sensor or a thermal sensor, wherein the processing circuit is further configured to receive data from the at least one of the infrared sensor or the thermal sensor.

11. The imaging device of claim 1, wherein the processing circuit is further configured to, using the neural network, learn a change and adapt the change into the library.

12. The imaging device of claim 1, wherein the neural network comprises a Radial Bases Function neural network.

13. The imaging device of claim 1, wherein, in an operational mode, the neural network is configured to provide a warning based on the comparison.

14. The imaging device of claim 1, wherein, in a learning mode, the neural network is configured to learn a new difference image.

15. The imaging device of claim 1, wherein, based on the comparison, if a difference between the first image and the first reference image is less than a threshold value, then the neural network learns a change and adapts the change into the library.

16. The imaging device of claim 1, wherein, based on the comparison, if a difference between the first image and the first reference image is greater than a threshold value, then the processing circuit transmits information of detection of debris to a user.

17. The imaging device of claim 1, wherein, based on the comparison, if a difference between the first image and the first reference image is greater than a threshold value, then the processing circuit transmits information of detection of at least one of an arc or a fire to a user.
